(19) United States
(12) Patent Application Publication
(10) Pub. No.: US 2021/0280290 A1
(43) Pub. Date: Sep. 9, 2021

(54) SYSTEM AND DEVICE TO MONITOR MULTIPLE INFUSIONS

(71) Applicant: KONINKLIJKE PHILIPS N.V., ElNDHOVEN (NL)

(72) Inventor: Mahmoudreza SHARIFI, New Rochelle, NY (US)

(21) Appl. No.: 17/261,368

(22) PCT Filed: Jul. 11, 2019
(86) PCT No.: PCT/EP2019/068752

§ 371 Date: Jan. 19, 2021

Related U.S. Application Data
(60) Provisional application No. 62/700,395, filed on Jul. 19, 2018.

(51) Int. Cl. G16H 20/17; G16H 40/40; G16H 50/70; G16H 40/20; G16H 40/67; G05B 19/042; A61M 5/172; G01F 9/00

(52) U.S. Cl. CPC G16H 20/17; G16H 40/40; G16H 50/70; G16H 40/20; G06K 1/123; G05B 19/042; A61M 5/172; G01F 9/003; G16H 40/67

(57) ABSTRACT

A method of administering a drug including measuring a dosage rate of a first intravenous (IV) bag connected to a first infusion pump, setting a pumping rate of the first infusion pump, comparing the pumping rate of the first infusion pump to the measured dosage rate of the first IV bag, and adjusting the pumping rate of the first infusion pump to the measured dosage rate of the first IV bag when the pumping rate of the first infusion pump is different from the measured dosage rate of the first IV bag.

### TECHNICAL FIELD

Various embodiments relate to medical systems and more particularly but not exclusively to monitoring systems of infusion pumps for administration of drugs to critically ill patients.

### BACKGROUND

Hospital patients such as intensive care unit (ICU) patients may receive multiple intravenous (IV) infusions concurrently. When a number of IV lines increase, tracking of the IV lines becomes complicated. In a stressful environment such as the ICU, the IV lines may be inadvertently mixed up. As a result, a patient can receive an incorrect type of medicine or the correct medicine at wrong rate or dosage, resulting in a medication error.

Medication errors are a significant concern because they can contribute to increased therapeutic complications of critically ill patients. Medication error is even riskier when it occurs in the ICU because a patient is more unstable in the ICU. Medication errors in this environment lead to a higher chance of catastrophe. Medication errors can lead to adverse drug events associated with deleterious outcomes for individuals and families and enormous economic burden on the healthcare system.

### SUMMARY

Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

The rest of the Summary section will track the claims when finalized.

### BRIEF DESCRIPTION OF THE DRAWINGS

Additional objects and features of the invention will be more readily apparent from the following detailed description and appended claims when taken in conjunction with the drawings. Although several embodiments are illustrated and described, like reference numerals identify like parts in each of the figures, in which:

FIG. 1 illustrates a system view of a drug delivery and monitoring system in accordance with embodiments described herein;

FIG. 2 illustrates a medical monitoring system 200 in accordance with FIG. 1.

FIG. 3 illustrates a block diagram of the central control unit 120 in accordance with FIG. 1; and

FIG. 4 illustrates a method of monitoring drug delivery of an IV bag in accordance with embodiments described herein.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or illustrated herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art, and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application. When steps of manufacture, process of using, or other method steps are described or claimed, the order of steps given is not constrained by the order presented, and may vary. Terms such as “below,” “above,” “right,” and “left,” may be used for relative orientation of a device or apparatus as illustrated in a figure. If an apparatus or component of a figure may be rotated and still function in a similar manner to what is described, the directional terms are not limited to the orientation illustrated in a particular figure. “Below” when rotated may become “right,” or “left” or “above.” The same holds true for the other directional indicators.

A desirable patient safety system in an ICU setting may incorporate several medication error prevention strategies at several phases of the medication use process. Medication errors occur in all types of medications. Because ICU medications are given through infusion pumps, errors of a wrong medicine or rate can be a source of error that is damaging or fatal to the patient.

Embodiments disclosed herein include a combination hardware and software solution to provide multiple checks of the medication and the infusion rate when there are several simultaneous infusions. According to embodiments described herein, errors due to mistakes by a bedside caregiver may be eliminated.

Embodiments described herein include a system that tracks medication from a time when prescribed to infusion of the medication to a patient. Embodiments include a method for automatically monitoring and setting the correct dosage rate for an intravenous (IV) device.

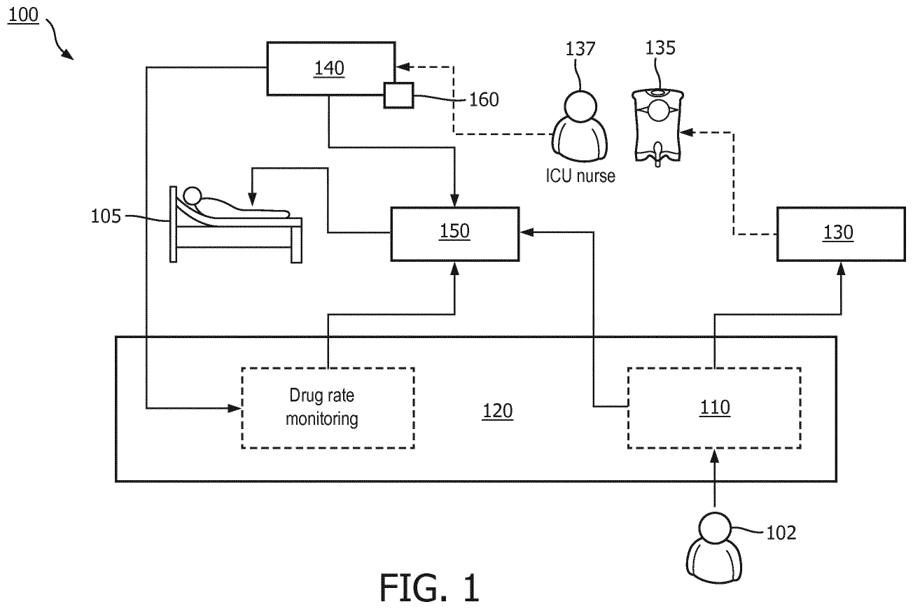

FIG. 1 illustrates a drug delivery and monitoring system 100 in accordance with embodiments described herein. In a medical setting, a clinician 102 may prescribe a plurality of drugs to be administered intravenously to a patient 105. The clinician 102 may enter a drug order by way of one or more prescriptions into a drug ordering system 110. The drug order is communicated by a central control unit 120 to a medication dispensing unit 130 which may print one or a plurality of coded (e.g., color coded) labels for IV bags. The labels may be affixed to IV bags and labeled IV bags 135 may be mounted to designated mounting apparatuses 140 such as hooks or a hanger by a medical worker 137 in the patient's room or place of treatment. The printed labels may indicate to the staff from which mounting apparatus 140 the labeled IV bags 135 are to be suspended. The mounting apparatuses 140 may also have a coding that matches the coding placed on the labeled IV bags 135 for easy matching of the labeled IV bags 135 to the proper mounting apparatus 140. After the medical staff hangs the labeled IV bags 135 on the correct mounting apparatuses 140, the staff connects other ends of one or more labeled IV bags 135 to one or a plurality of infusion pumps 150. The infusion pumps 150 may be controlled over a network by the central control unit 120.

Different procedures may be undertaken by medical staff when setting up IV infusion for a patient or patients. Initially, a doctor may examine a new ICU patient's file to decide which drugs to prescribe, based on inputs from medical staff such as a surgeon and anesthetist. The doctor may enter all prescriptions into an Electronic Patient Record (EPR) that is stored in the central control unit 120 or in another database. Using patient-specific parameters such as body weight, the central control unit 120 may calculate the required medication dose and flow rate based on medication information available in a drug chart menu of the EPR. A nurse or other qualified medial staff may then prepare and administer the required drugs to the patient.

A nurse or other qualified medical worker may connect the IV bag to the infusion pump 150 and to the patient, programming the infusion rate, and optionally volume to be infused. After the infusion is started, the central control unit 120 periodically monitors the weight of the bag and the volume infused. The data may be recorded into the EPR or stored elsewhere in the system. A new bag is replaced when required.

The drug delivery and monitoring system 100 enables the central control unit 120 to check levels of one or more IV bags based on the weight of an IV bag and its contents. Knowing the initial weight of an IV bag and its contents, embodiments described herein monitor the weight of the contents at different intervals. Based on a change in weight over time, the central control unit 120 calculates and determines an amount of fluid present and the rate of dispensing of the fluid per minute or other interval. The central control unit 120 may send out alerts or alarms via SMS or e-mail based on limits set by an authorized administrator. When connected to a mounting apparatus 140, the labeled IV bags 135 may suspend from a load cell 160 or other weight sensor that is part of or connects to the mounting apparatus 140. The load cell 160 is able to detect changes in weight of the labeled IV bags 135 and send feedback to the central control unit through the network interface. The components described in FIG. 1 may be controlled and monitored by the central control unit 120. The central control unit 120 uses components of the drug delivery and monitoring system 100 in a feedback control loop to ensure that a correct labeled IV bag 135 is connected to a correct network-controlled infusion pump 150 and is being infused into a patient at a correct rate.

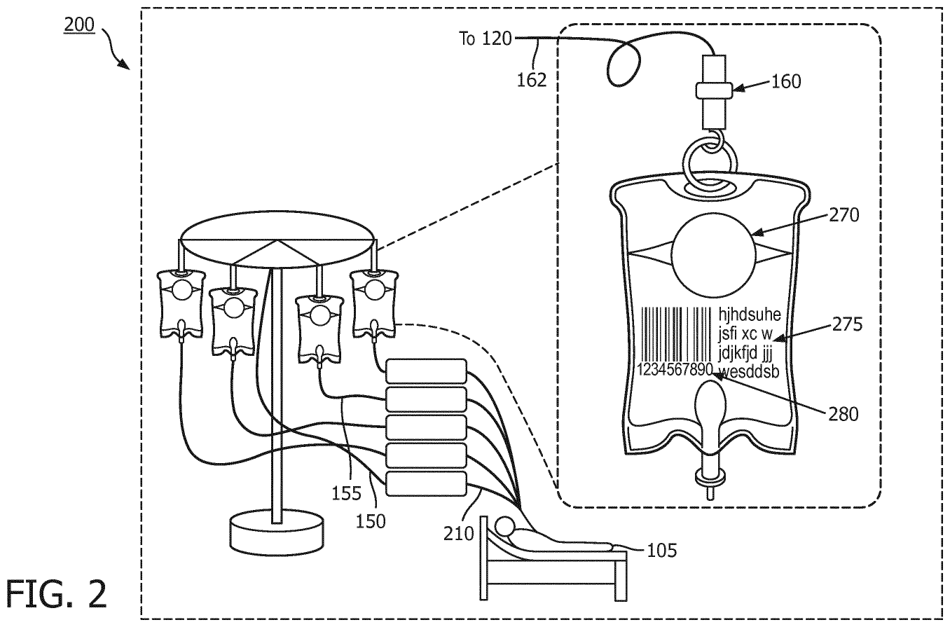

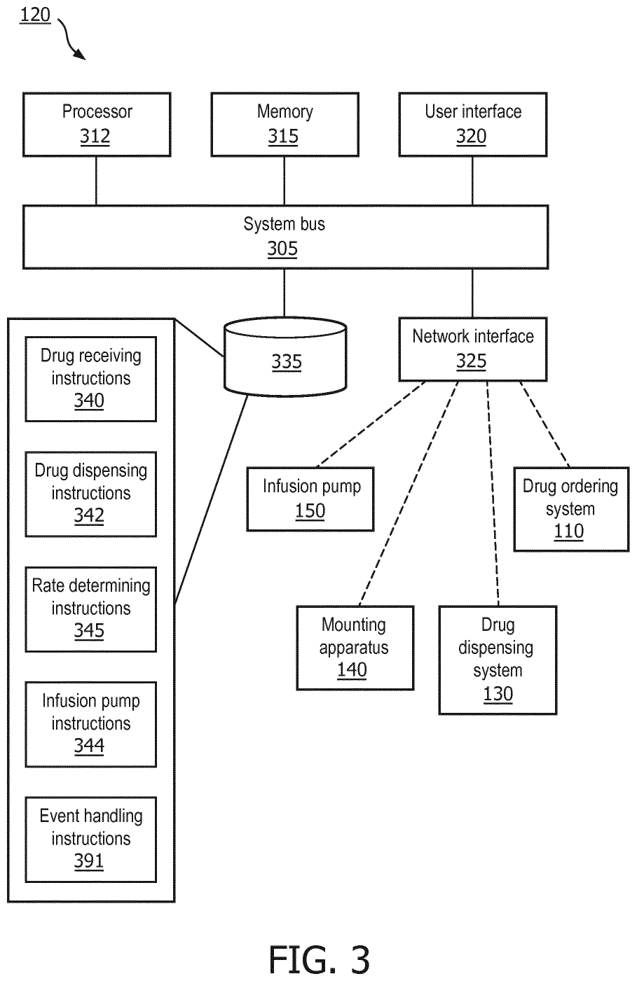

FIG. 2 illustrates a medical monitoring system 200 in accordance with FIG. 1. A patient 105 may be stationed on a bed or other apparatus such as a chair. Multiple infusion lines 210 may be connected to the patient. The multiple infusion lines 210 may each be connected to separate infusion pumps 150. Separately labeled IV bags 135 may be respectively connected to various infusion pumps 150 through IV tubing 155. Each labeled IV bag 135 may hang from a hook or other mounting apparatus 140 that includes the load cell 160. The load cell 160 may be wired or wirelessly connected 162 to the central control unit 120. The labeled IV bag may have multiple forms of data identification such as a color label 270, written medication information 275, and/or a bar code label 280. The infusion pumps 150 may be wired or wirelessly connected to the central control unit 120. The central control unit 120, after receiving medication instructions from a medical practitioner, may set desired pumping rates of the infusion pumps 150 to correspond with the labeled IV bags 135 as described herein. Components of the drug delivery and monitoring system will be further described with reference to FIG. 3.

FIG. 3 illustrates a block diagram of the central control unit 120 in accordance with FIG. 1. The central control unit 120 includes a processor 312, memory 315, network interface 325, and storage 335 interconnected via one or more system buses 305. The system bus 305 may operate in a wired or wireless manner to transfer data between components in the drug delivery and monitoring system 100.

The storage 235 of the central control unit 120 may include various instructions for achieving the functionality described herein. For example, the storage 335 may include drug input instructions 340 for determining prescriptions input by medical workers. The prescriptions may be input by various medical personnel such as doctors, nurses, nurse practitioners, etc. at one or more drug ordering systems 110 located in a medical facility or remote therefrom. Such prescriptions may be stored in the storage 335 as a catalog of prescriptions that may be accessed at any time.

The storage 335 may include drug dispensing instructions 342. The drug dispensing instructions 342 may include dosage and labeling information that may be sent to a medication dispensing unit 130 that is configured to output labels for IV bags. Labels may be printed and affixed to IV bags or fully labeled IV bags indicating usage information for the medicine contained therein may be dispensed from the medication dispensing unit 130. The drug dispensing instructions 342 may access look-up tables in storage 335 that match medication names to labeling schemes, and send those labeling schemes to the medication dispensing unit 130.

Infusion pump instructions 344 stored in the storage 335 may direct one or more infusion pumps 150 to operate at a particular infusion rate to coincide with the medication prescribed by a medical professional. Rate determining instructions 345 receive feedback weight measurements from the load cell 160 and use those measurements to determine a dosage rate based upon weight changes over time. When the infusion pump 150 is connected to the labeled IV bag 135 and the system begins pumping, the rate determining instructions 345 may receive feedback weight information from the load cell 160 at different time periods. A weight per time period calculation may be performed to determine the dosage rate for the drug in the labeled IV bag 135. This calculated dosage rate may be compared with the commanded dosage rate for the infusion pump 150 associated with the labeled IV bag 135 to determine if the labeled IV bag 135 is connected to the wrong port of an infusion pump.

The central control unit 120 may direct set up of a first labeled IV bag 135 with a first color code to be dispensed at a first rate by a first pump. If the labeled IV bag 135 is connected to a second pump by mistake, the second pump will pump the first IV bag at the second rate. In another embodiment, the labeled IV bag 135 may be pumped at an incorrect rate by the first pump. Because the central control unit 120 knows the first rate at which the first IV bag is supposed to pump, when the load cell 160 sends weight measurements over a predetermined time period, the dosage rate of the first IV bag will be compared with the expected first infusion rate. If the measured rate and the expected first infusion rate are the same, the first IV pump will continue pumping at the first rate. If the measure rate and the expected first infusion rate are not the same, the central control unit 120 will determine the correct rate for the first labeled IV bag, and adjust the infusion pump connected to the first labeled IV bag to pump at the first rate.

The load cell 160 may reflect the change in weight and the central control unit 120 may compute the rate and compare it to the rate of the infusion pump 150. This weight change of the load cell 160 is one form of feedback and can be monitored by the central control unit 120 as a secondary check to determine if a first IV bag is being infused at the correct weight. When an error occurs, the central control unit 120 determines the correct rate of the errant infusion pump 150, and adjusts the rate to match the infusion rate indicated by the load cell that labeled IV bag 135 that are connected thereto.

In one embodiment, one or more infusion pumps may be stopped or paused to determine if connections to IV bags and pumping rates are correct. For example, if a first infusion pump is connected to a first IV bag that is connected to a first load cell, the first infusion pump may be temporarily stopped for a predetermined amount of time. After the predetermined amount of time, the central control unit 120 may check signals sent by the first load cell to determine if the weight per unit time of the first load cell is still changing. If the first load cell has stopped changing weight, the central control unit 120 may determine that the first infusion pump is correctly connected to the first IV bag and first load cell. If the first load cell continues to register a change in weight per unit time, the central control unit 120 may determine that the first load cell and first infusion pump are not correctly connected. The central control unit 120 may then query the other load cells to see which of them shows no change in weight, which could indicate that the associated IV bag may be connected to the stopped pump. The central control unit 120 may then systematically stop additional infusion pumps pumping fluids for a patient and conduct similar measurements of the first load cell to determine when the first load IV bag ceases to change in weight. When a correct match between the measurements regarding the first load cell and the correct infusion pump are determined, the pumping rate of the correct infusion pump may be adjusted by the central control unit 120 to match the desired pumping rate of the first IV bag. Similar procedures may be conducted for other IV bags in a system.

The storage 335 may include event handling instructions 391 for receiving and interpreting events, such as mismatched IV bags. When an IV bag of a certain color, identification, and rate is connected to a correct port of an infusion pump 150, the drug delivery and monitoring system 100 operates as normal. When an IV bag is connected to a wrong pump that begins to pump the IV bag at a different rate than expected, the event handling instructions 391 may cause the infusion rate of the infusion pump to be adjusted to run at the correct dosage rate.

The mounting apparatus 140 having a load cell 160 attached thereto may include weight sensors to receive multiple IV bags and a network interface to send data to the central control unit 120.

In one embodiment, the infusion pump 150 may be a volumetric pump that delivers drugs to patients from external bags that contain a volume of medication. The central control unit 120 sets an initial rate of the infusion pump(s), monitors the weights of the bag(s) via the load cell(s) 160, and uses feedback from the load cells to adjust the rates of the infusion pumps to achieve the prescribed rate.

The infusion pump 150 may be controlled by the central control unit 120 through the network interface 325 and may include a processor, memory, user interface, electronic pump, network interface, and storage interconnected via one or more system buses. The infusion rate may be regulated by the electronic pump to deliver fluids at the correct rate and volume. IV pumps may regulate the rate of fluids in ml/hr.

It will be understood that FIG. 3 constitutes, in some respects, abstractions and that the actual organization of the components of the devices and systems 110, 120, 130, 140, and 150 may be more complex than illustrated.

The processor 312 may be any hardware device capable of executing instructions stored in the memory 315 or storage 335 or otherwise processing data. As such, the processor 312 may include a microprocessor, field programmable gate array (FPGA), application-specific integrated circuit (ASIC), programmable logic controller (PLC), or other similar devices.

The memory 315 may include various memories such as, for example L1, L2, or L3 cache or system memory. As such, the memory 315 may include static random access memory (SRAM), dynamic RAM (DRAM), flash memory, read only memory (ROM), or other similar memory devices.

The user interface 320 may include one or more devices for enabling communication with a user. For example, the user interface 320 may include a display, a touchscreen, a mouse, and/or a keyboard for receiving user commands. In some embodiments, the user interface 220 may include a command line interface or graphical user interface that may be presented to a remote terminal via the network interface 325.

The network interface 325 may include one or more devices for enabling communication with other hardware devices. For example, the network interface 325 may include a network interface card (NIC) configured to communicate according to the Ethernet protocol or WiFi protocol. Additionally, the network interface 325 may implement a TCP/IP stack for communication according to the TCP/IP protocols. Various alternative or additional hardware or configurations for the network interface 325 will be apparent. As shown, the network interface 325 are configured to communicate with each other via, for example, a WiFi or Ethernet connection.

The storage 335 may include one or more machine-readable storage media such as read-only memory (ROM), random-access memory (RAM), magnetic disk storage media, optical storage media, flash-memory devices, or similar storage media. In various embodiments, the storage 335 may store instructions for execution by the processor 312 or data upon with the respective processor 312 may operate.

It will be apparent that various information described as stored in the storage 335 may be additionally or alternatively stored in the memory 315. In this respect, the memory 315 may also be considered to constitute a “storage device” and the storage 335 may be considered a “memory.” Various other arrangements will be apparent. Further, the memory 315 and storage 335 may both be considered to be “non-transitory machine-readable media.” As used herein, the term “non-transitory” will be understood to exclude transitory signals but to include all forms of storage, including both volatile and non-volatile memories.

The processor 312 may include multiple microprocessors that are configured to independently execute the methods described herein or are configured to perform steps or subroutines of the methods described herein such that the multiple processors cooperate to achieve the functionality described herein.

In this system, once a new medication is prescribed, the information is directly sent by the central control unit to both the infusion pumps 150 and a drug dispensing system. Based on this information, the drug dispensing unit 130 color codes the IV bag or an IV bag label and the care giver uses the color code to hang the IV bag at the right hanger. Each hanger is equipped with a sensitive load cell 160 to monitor the weight and the IV bag and the rate of the change of this weight which is the same as infusion rate. This information along with the real time of the medication is then sent back to the central control unit 120 where the information is double checked with the prescription schedule.

The use of technology including computerized prescriber order entry (CPOE), clinical decision support systems (CDSS), bar-coded medication administration (BCMA) systems, and smart IV infusion pumps can minimize the risk of error. Also, implementing new practices such as medication reconciliation and standardized IV medication concentration practices may reduce medication errors.

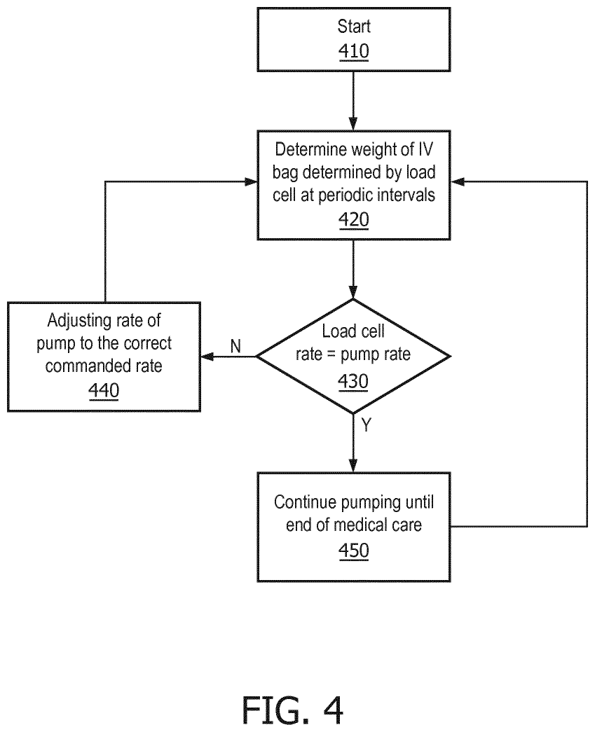

FIG. 4 illustrates a method of monitoring drug delivery of an IV bag in accordance with embodiments described herein. The proposed method includes steps to monitor the rate of one or more drugs received by a patient. Starting point 410, represents when a correctly labeled IV bag 135 has been connected to a matching mounting apparatus. The labeled IV bag 135 is then connected to an infusion pump and to a patient. At step 420, a load cell 160 begins to measure a weight of the labeled IV bag 135 at different intervals and sends this data to the central control unit 120. At step 430, the central control unit measures a gradual change in the weight of the IV fluid, hence the actual rate of the drug being administered.

During drug infusion, the load cell 160 will continuously measure the weight 420 of each IV bag so the central control unit 120 can detect the rate of the change of the infusion. At step 430, the system determines if the infusion rate indicated by each central control unit 120 is equal to the commanded pump rate associate with each IV bag. If the infusion rates of one or more IV bags are not equal to the correct infusion rate that is prescribed for each associated infusion pump, the central control unit may detect the mismatch and adjust the infusion rates at step 440 of any infusion pumps that do not match the dosage of the associated IV bags. After adjustment at step 440, the method returns to step 420 to measure the weight of the IV bags. If the method is unable to correctly set the pump infusion rate, a warning signal may be sent to the caregiver to check if there is any problem. Once the load cell rate equals the pump rate the method continues to pump until the end of medical care 450 and returns to step 420 periodically to ensure that the correct drug infusion rate is maintained for each IV bag.

The method described herein does not require the central control unit 120 to know which IV bag is connected to which pump. The feedback from the load cell 160 allows adjustment of all infusion pumps 150 in the room to the correct rate on the assumption that the staff successfully matched the bag code to the hook code.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be effected while remaining within the spirit and scope of the invention. Accordingly, the foregoing disclosure, description, and figures are for illustrative purposes only and do not in any way limit the invention, which is defined only by the claims.

1. A method of adjusting a pumping rate of an infusion pump comprising:
receiving a setting for a pumping rate of a first infusion pump;
measuring a dosage rate of a first intravenous (IV) bag connected to the first infusion pump based upon a change in weight of the first IV bag;
comparing the measured dosage rate of the first IV bag to a first prescribed infusion rate; and
wherein when the measured dosage rate of the first IV bag is different to the first prescribed infusion rate, performing the steps of:
(a) adjusting the pumping rate of the first infusion pump;
(b) measuring the dosage rate of the first IV bag; and
(c) comparing the measured dosage rate of the first IV bag to the first prescribed infusion rate, and
repeating steps (a)-(c) until the measured dosage rate of the first IV bag is the same as the first prescribed infusion rate.

2. The method of claim 1, wherein the dosage rate of the first IV bag is determined at periodic intervals.

3. (canceled)

4. The method of claim 1, comprising:
receiving prescription information for a patient; and
sending the prescription information to a drug dispensing system to prepare contents of the first IV bag.

5. (canceled)

6. The method of claim 1, comprising controlling a delivery of medication information to a drug dispensing system.

7. The method of claim 1, comprising:
receiving a setting for a pumping rate of a second infusion pump;
measuring a dosage rate of a second intravenous (IV) bag connected to the second infusion pump based upon a change in weight of the second IV bag;
comparing the measured dosage rate of the second IV bag to a second prescribed infusion rate; and
wherein when the measured dosage rate of the second IV bag is different to the second prescribed infusion rate, performing the steps of:
(a) adjusting the pumping rate of the second infusion pump;
(b) measuring the dosage rate of the second IV bag;
(c) comparing the measured dosage rate of the second IV bag to the second prescribed infusion rate, and
repeating steps (a)-(c) until the measured dosage rate of the second IV bag is the same as the second prescribed infusion rate.

8. A non-transitory machine-readable storage medium encoded with instructions for adjusting a pumping rate of an infusion pump, comprising:
instructions for setting a pumping rate of a first infusion pump;
instructions for measuring a dosage rate of a first intravenous (IV) bag connected to the first infusion pump based upon a change in weight of the first IV bag;
instructions for comparing the measured dosage rate of the first IV bag to a first prescribed infusion rate; and
instructions for, wherein when the measured dosage rate of the first IV bag is different to the first prescribed infusion rate, performing the steps of:
(a) adjusting the pumping rate of the first infusion pump;
(b) measuring the dosage rate of the first IV bag;
(c) comparing the measured dosage rate of the first IV bag to the first prescribed infusion rate, and
repeating steps (a)-(c) until the measured dosage rate of the first IV bag is the same as the first prescribed infusion rate.

9. The non-transitory machine-readable storage medium of claim 8, wherein the dosage rate of the first IV bag is determined at periodic intervals.

10. (canceled)

11. The non-transitory machine-readable storage medium of claim 8, comprising instructions for receiving prescription information for:
receiving prescription information for a patient; and
sending the prescription information to a drug dispensing system to prepare contents of the first IV bag.

12. (canceled)

13. The non-transitory machine-readable storage medium of claim of claim 8, comprising instructions for controlling a delivery of medication information to a drug dispensing system.

14. The non-transitory machine-readable storage medium of claim 7, comprising:
instructions for setting a pumping rate of a second infusion pump;
instructions for measuring a dosage rate of a second intravenous (IV) bag connected to a the second infusion pump based upon a change in weight of the second IV bag;
instructions for comparing the measured dosage rate of the second IV bag to a second prescribed infusion rate; and
instructions for, wherein when the measured dosage rate of the second IV bag is different to the second prescribed infusion rate, performing the steps of:
(a) adjusting the pumping rate of the second infusion pump;
(b) measuring the dosage rate of the second IV bag;
(c) comparing the measured dosage rate of the second IV bag to the second prescribed infusion rate, and
repeating steps (a)-(c) until the measured dosage rate of the second IV bag is the same as the second prescribed infusion rate.

15. A system for administering a drug, comprising:
a memory to store instructions for interacting with and acquiring data from a plurality of drug administering sub-systems;
a processor for processing the data acquired from one or a plurality of drug administering sub-systems; and
a network interface to connect the memory and processor to the plurality of drug administering sub-systems,
wherein the processor is configured to measure a dosage rate of an intravenous (IV) bag based upon a change in weight of the IV bag, compare the dosage rate to a prescribed infusion rate, and alter the infusion rate of an infusion pump when the dosage rate of the IV bag is different to the prescribed infusion rate.

16. The system of claim 15, wherein the processor receives drug ordering instructions from a drug ordering system.

17. The system of claim 15, wherein the processor dispenses medication information to a drug dispensing system.

18. The system of claim 15, wherein the dosage rate of the IV bag is determined at periodic intervals.

19. (canceled)

20. The system of claim 15, wherein the process is configured to turn off the infusion pump for a predetermined period and to compare the dosage rate of the IV bag to the infusion rate of the infusion pump to determine whether the IV bag is connected to the infusion pump.
